(19) United States
(12) Patent Application Publication
(10) Pub. No.: US 2017/0273889 A1
(43) Pub. Date: Sep. 28, 2017

(54) AGENT AND METHOD FOR THE TEMPORARY SHAPING OF KERATIN-CONTAINING FIBERS

(71) Applicant: Henkel AG & Co. KGaA, Duesseldorf (DE)

(72) Inventors: Thorsten Knappe, Schenefeld (DE); Thomas Foerster, Duesseldorf (DE); Rolf Bayersdoerfer, Hamburg (DE)

(73) Assignee: Henkel AG & Co. KGaA, Duesseldorf (DE)

(21) Appl. No.: 15/531,494

(22) PCT Filed: Nov. 2, 2015
(86) PCT No.: PCT/EP2015/075378

§ 371 Date: May 30, 2017

(30) Foreign Application Priority Data
Dec. 10, 2014 (DE) 10 2014 225 442.6

(51) Int. Cl. A61K 8/73; A61Q 5/06; A45D 34/04; A61K 8/86; A61K 8/92; A61K 8/81; A61K 8/34

(52) U.S. Cl. CPC A61K 8/736; A61K 8/8182; A61Q 5/06; A61K 8/34; A61K 8/86; A61K 8/922; A45D 34/04; A61K 2800/805; A61K 2800/5422; A61K 2800/5426; A61K 2800/87; B05B 9/002

(57) ABSTRACT

The disclosure relates to a cosmetic product, comprising a) a cosmetic preparation, containing, in relation to its total weight, a1) 45 to 98 wt.-% of at least one polar solvent; a2) 0.1 to 4.0 wt.-% of chitosan; and a3) 0.1 to 15 wt.-% of a film-forming polymer; b) a device for flash evaporation of the cosmetic preparation a), and to a method using corresponding products and the use of the cosmetic preparation a) as process material in a device for flash evaporation.

### CROSS-REFERENCE TO RELATED APPLICATION

The application is a U.S. National-Stage entry under 35 U.S.C. §371 based on International Application No. PCT/EP2015/075378, filed Nov. 2, 2015 which was published under PCT Article 21(2) and which claims priority to German Application No. 10 2014 225 442.6, filed Dec. 10, 2014, which are all hereby incorporated in their entirety by reference.

### TECHNICAL FIELD

The present disclosure relates to the technical field of temporary shaping of keratin-containing fibers, in particular human hair. The subject matter of the disclosure is specific cosmetic hair formulations which are suitable for application to keratin-containing fibers by employing a flash evaporation process. In addition, the use of these cosmetic hair formulations in devices for flash evaporation and methods for temporary shaping of keratin-containing fibers are the subject matter of the present disclosure.

### BACKGROUND

An attractive-looking hairstyle is considered in general to be an essential part of a well-groomed appearance today. Because of current fashion trends, hairstyles that can be created and/or maintained for a longer period of time lasting up to several days are possible with many types of hair only by using active ingredients that have a hair setting effect. Therefore, hair treatment agents, which serve to provide a permanent or temporary shape to hair play an important role. Whereas the chemical structure of the keratin-containing fibers is modified by reduction and oxidation processes in permanent shaping, such modifications in the chemical structure do not take place with temporary shaping. Corresponding agents for temporary shaping usually contain synthetic polymers and/or waxes as the active hair-setting ingredient.

The most important property of an agent for temporary shaping of keratin-containing fibers, hereinafter referred to as styling agents, consists of imparting the strongest possible hold to the treated fibers in the newly modeled form—i.e., a form imposed on the fibers. If the keratin-containing fibers are human hair, then we also speak of a strong hairstyle hold or a high degree of hold of the styling agent. The hairstyle hold is determined essentially by the type and amount of active hair-setting ingredients used, but there may also be an influence of the other ingredients of the styling agent as well as the form of application.

In the field of temporary shaping of keratin-containing fibers, spray application of corresponding cosmetic preparations plays an important role in particular, wherein these preparations are usually applied as pump sprays or aerosol sprays. To do so, the cosmetic preparations are finished in a dispensing device from which they are sprayed either by application of a mechanical force or with the help of a blowing agent through a valve. Both methods have obvious disadvantages. Whereas pump sprays are not usually suitable for a uniform and long-lasting spray application of cosmetic hair preparations, aerosol sprays are based on the use of blowing agents or blowing agent gases, which do not manifest a cosmetic effect and, on the other hand, may result in a risk to the consumer if handled incorrectly.

Against this background, there is a demand for alternative methods of atomizing cosmetic hair preparations. One such alternative spray method has proven to be flash evaporation. In this method, which is described in the International Patent Application WO 2001/83071 A1 (Henkel) for example, a solvent-based liquid or pasty composition is heated in a closed space to a temperature above the boiling point of the solvent, so that an excess pressure is generated in the composition. On depressurization (throttling) of the pressure, the liquid evaporates and can subsequently be atomized by employing a suitable nozzle, for example.

Although flash evaporation is fundamentally suitable for spray application of cosmetic hair preparations, at the same time not all cosmetic hair preparations can be atomized by employing a flash evaporation method. This is due on the one hand to the heating of the cosmetic preparation required for flash evaporation and on the other hand to the specific properties of the spray mist formed by flash evaporation, for example, the size of the droplets created and the droplet density in the spray mist.

### BRIEF SUMMARY

A cosmetic product is provided herein. The cosmetic product includes a cosmetic preparation. The cosmetic preparation includes, based on its total weight, about 45% to about 98% by weight of at least one polar solvent, about 0.1% to about 4.0% by weight chitosan, and about 0.1% to about 15% by weight film-forming polymer. The cosmetic product further includes a device for flash evaporation of the cosmetic preparation.

A cosmetic preparation is also provided herein. The cosmetic preparation includes, based on its total weight, about 45% to about 98% by weight of at least one polar solvent, about 0.1% to about 4.0% by weight chitosan, and about 0.1% to about 15% by weight film-forming polymer. The cosmetic product further includes a device for flash evaporation of the cosmetic preparation. The cosmetic preparation is utilized as a material in a device for flash evaporation.

A method for temporary shaping of keratin-containing fibers is also provided herein. The method includes treating the keratin-containing fibers with a cosmetic preparation by employing a device for flash evaporation. The cosmetic preparation includes, based on its total weight, about 45% to about 98% by weight of at least one polar solvent, about 0.1% to about 4.0% by weight chitosan, and about 0.1% to about 15% by weight film-forming polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to make available specific cosmetic hair preparations for temporary shaping of keratin-containing fibers, which will be suitable because of their chemical and physical properties for targeted spray application by employing a device for flash evaporation. In addition, the preparations should also be suitable for implementing a high degree of hold, in particular a long-lasting hold after application by employing a flash evaporation method and for implementing a high-volume effect. It has been found that, from the variety of known cosmetic hair polymer preparations, to solve this problem, solvent-containing preparations based on a combination of film-forming polymers containing chitosan and its derivatives are suitable in particular.

The first subject matter of the present disclosure is thus a cosmetic product, comprising:
a) a cosmetic preparation containing, based on its total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a).

The cosmetic preparation a) is preferably liquid. The cosmetic preparation a) may be in the form of a solvent or dispersion, for example, an emulsion or suspension. Preferred cosmetic preparations a) are in the form of a solution or a suspension.

The cosmetic preparation as contemplated herein contains as the first essential component about 45% to about 98% by weight of at least one polar solvent a1). Preferred cosmetic products are characterized in that the amount by weight of the polar solvent a1) in the total weight of the cosmetic preparation a) is about 55% to about 95% by weight, preferably about 70% to about 92% by weight. Corresponding agents are characterized by a good cosmetic effect with good applicability at the same time.

To improve the application properties of cosmetic preparations as contemplated herein, while at the same time minimizing the thermal load of any active ingredients or excipients in the course of the flash evaporation process, it has proven advantageous to use polar solvents a1), which have a boiling point (20° C., 1013 mbar) between about 50° C. and about 110° C., preferably between about 70° C. and about 105° C. Ethanol, isopropanol and water have proven to be particularly suitable here and are therefore preferred as polar solvent a1) for this reason.

Particularly preferred polar solvents a1) or solvent systems are

characterized in that the amount by weight of water and ethanol in the total weight of the polar solvent a1) is at least about 60% by weight, preferably at least about 80% by weight, especially preferably at least about 90% by weight, and in particular at least about 95% by weight;

characterized in that the amount by weight of water in the total weight of the polar solvent a1) is more than about 80% by weight, preferably more than about 88% by weight and in particular more than about 92% by weight.

Most especially preferred cosmetic preparations are characterized in that the amount of volatile components is no greater than about 55% by weight. The polar solvent ethanol is also included in the group of these volatile components. Most especially preferred cosmetic preparations are thus in other words, characterized in that the amount by weight of ethanol in the total weight of the cosmetic preparation amounts to no greater than about 55% by weight, preferably about 10% to about 55% by weight, especially preferably about 25% to about 55% by weight, and in particular about 40% to about 55% by weight.

A second important ingredient of cosmetic compositions as contemplated herein is chitosan a2). With respect to the manufacturability, applicability and cosmetic effect of cosmetic compositions of the disclosure, it has proven to be advantageous if the amount by weight of chitosan a2) in the total weight of the cosmetic preparation a) amounts to about 0.1% to about 2.0% by weight, preferably about 0.2% to about 1.0% by weight.

Chitosans are biopolymers and are included in the group of hydrocolloids. From a chemical standpoint, these are partially deacetylated chitins of different molecular weights.

To produce the chitosans, one starts with chitin, preferably the shell residues of crustacean creatures that are available in large quantities as an inexpensive raw material. Chitin is usually deproteinized first by adding bases, then demineralized by adding mineral acids and finally deacetylated by adding strong bases, while the molecular weights may be distributed over a broad spectrum. Such molecular weights as an average molecular weight (weight average) of about 800,000 to about 1,200,000 Dalton, a viscosity according to Brookfield (1% by weight solution in glycolic acid) of less than about 5000 mPas, a degree of deacetylation in the range of about 80% to about 88% and an ash content of less than about 0.3% by weight are preferably used.

In addition to the chitosans as typical biopolymers, cationically derivatized chitosans (for example, quaternization products) or alkoxylated chitosans may also be used in the sense of the present disclosure as derivatives of chitosan.

Preferred agents as contemplated herein are characterized in that they contain as the chitosan at least one neutralization product of chitosan with at least one organic carboxylic acid, such as in particular formic acid, acetic acid, citric acid, lactic acid, pyrrolidone carboxylic acid, tartaric acid, glycolic acid, nicotinic acid, hydroxyisobutyric acid, hydroxyisovaleric acid or mixtures of these acids. It is preferred as contemplated herein to select the organic carboxylic acid from lactic acid, formic acid, pyrrolidone carboxylic acid, nicotinic acid, hydroxyisobutyric acid, hydroxyisovaleric acid or mixtures of these acids. This neutralization product may be prepared for example, in an aqueous medium by adding chitosan and the corresponding carboxylic acid.

Suitable chitosans that are freely available commercially include, for example, those available under the brand names Hydagen® CMF (1% by weight active substance in aqueous solution with 0.4% by weight glycolic acid, molecular weight about 500,000 to about 5,000,000 g/mol, Cognis), Hydagen® HCMF (chitosan 80% deacetylated, molecular weight about 50,000 to about 1,000,000 g/mol, Cognis), Kytamer® PC (80% by weight active substance on chitosan pyrrolidone carboxylate (INCI designation: chitosan PCA), Amerchol) and Chitolam® NB/101.

A third important component of the cosmetic preparations a) as contemplated herein is the film-forming polymers a3). Suitable film-forming polymers a3) include permanently as well as temporarily cationic, anionic, nonionic or amphoteric polymers. These film-forming polymers may be of a synthetic or natural origin. Preferred cosmetic preparations a) contain, based on their total weight, about 1.0% to about 12% by weight, preferably about 2.0% to about 10% by weight film-forming polymer a3). This polymer a3) is different from chitosan a2).

Examples of conventional film-forming polymers a4) include acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C₁₋₂ succinates/hydroxy acrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethyl acrylate phosphate/acrylates copolymer, aminoethylpropanediol-acrylates/acrylamide copolymer, aminoethylpropanediol-AMPD-acrylates/diacetone acrylamide copolymer, ammonium VA/acrylates copolymer, AMPD acrylates/diacetone acrylamide copolymer, AMP acrylates/allyl methacrylate copolymer, AMP acrylates/C₁₋₁₈ alkyl acrylates/C₁₋₁₈ alkyl acrylamide copolymer, AMP-acrylates/diacetone acrylamide copolymer, AMP acrylates/dimethylaminoethyl methacrylate copolymer, bacillus/rice bran extract/soybean extract ferment filtrate, bis-butyloxyamodimethicone/PEG-60 copolymer, butyl acetate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, cornstarch/acrylamide/sodium acrylate copolymer, diethylene glycolamine/epichlorohydrin piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutyl methacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, poly-beta-alanine/glutaric acid crosspolymer, polybutylene terephthalate, polyester-1, polyethyl acrylate, polyethylene terephthalate, polymethacryloyl ethylbetaine, polypentaerythrityl terephthalate, polyperfluoro perhydrophenanthrene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-39, polyquaternium-45, polyquaternium-46, polyquaternium-47, polyquaternium-48, polyquaternium-49, polyquaternium-50, polyquaternium-55, polyquaternium-56, polyquaternium-68, polyquaternium-69, polysilicone-9, polyurethane-1, polyurethane-6, polyurethane-10, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVPNA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethyl methacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

A group of polymers a3) preferably used in the cosmetic preparation includes the vinylpyrrolidone homopolymer or copolymer B. Polymers a3) that are especially preferably used include, for example:

polyvinylpyrrolidones, such as those distributed under the brand name Luviskol® (BASF), for example,

vinylpyrrolidone/vinyl ester copolymers, such as those distributed under the brand name Luviskol® (BASF), for example; Luviskol® VA 64 and Luviskol® VA 73, each of which are vinylpyrrolidone/vinyl acetate copolymers, are preferred nonionic polymers.

Because of their cosmetic effect in combination with chitosan a2), film-forming polymers that are preferably used as contemplated herein include in particular the polyvinylpyrrolidones (INCI designation: PVP) and the polyvinylpyrrolidone/vinyl acetate copolymers (INCI designation: VP/VA copolymer). Preferred cosmetic products are characterized in that the film-forming polymer a3) is selected from the group of nonionic polymers, preferably from the group of polyvinylpyrrolidones and vinylpyrrolidone/vinyl acetate copolymers, preferably the vinylpyrrolidone/vinyl acetate copolymers.

Another group of preferred film-forming polymers a3) is formed by the cationic polymers, in particular cationic polymers with the INCI designations polyquaternium-1, polyquaternium-2, polyquaternium-4, polyquaternium-5, polyquaternium-6, polyquaternium-7, polyquaternium-8, polyquatemium-9, polyquatemium-10, polyquatemium-11, polyquaternium-12, polyquaternium-13, polyquatemium-14, polyquaternium-15, polyquaternium-16, polyquaternium-17, polyquatemium-18, polyquaternium-19, polyquaternium-20, polyquaternium-22, polyquatemium-24, polyquaternium-27, polyquaternium-28, polyquaternium-29, polyquatemium-30, polyquaternium-31, polyquaternium-32, polyquaternium-33, polyquatemium-34, polyquaternium-35, polyquaternium-36, polyquaternium-39, polyquatemium-45, polyquaternium-46, polyquaternium-47, polyquaternium-48, polyquatemium-49, polyquaternium-50, polyquatemium-55, polyquatemium-56, polyquatemium-68 and polyquaternium-69. Most especially preferred cosmetic products are characterized in that the film-forming polymer a3) is selected from the group of cationic polymers with the INCI designations polyquaternium-4, polyquaternium-11, polyquaternium-16, polyquaternium-55, polyquaternium-69. Corresponding cosmetic products are characterized by particularly good cosmetic hair properties, in particular care and styling properties.

A first group of especially preferred cationic polymers a3) include the cationic cellulose derivatives, in particular those derived from a reaction of hydroxyethyl cellulose with a dimethyldiallylammonium reactant (in particular dimethyldiallylammonium chloride), optionally in the presence of additional reactants. Of these cationic celluloses, the cationic celluloses with the INCI designation polyquaternium-4 are especially preferred. Corresponding celluloses are distributed under the designations Celquat® H 100 and Celquat® L 200, for example, by the National Starch company.

A second group of especially preferred cationic polymers a3) are the copolymers of vinylpyrrolidone with quaternized derivatives of dialkylaminoalkyl acrylate and methacrylate, in particular copolymers of vinylpyrrolidone and dimethylaminoethyl methacrylate quaternized with diethyl sulfate (INCI designation: polyquaternium-11). Corresponding polymers can be obtained under the brand names Gafquat® 440, Gafquat® 734 and Gafquat® 755 N (ISP), for example.

Other preferred cationic polymers a3) include N-methylvinylimidazole/vinylpyrrolidone copolymers (INCI designation: polyquaternium-16), which are distributed by BASF, for example, under the brand names Luviquat® Style, Luviquat® FC 370, Luviquat® FC 550, Luviquat® FC 905 and Luviquat® HM 552.

In addition, copolymers of methacryloylaminopropyl lauryldimethyl ammonium chloride with vinylpyrrolidone and methaylaminopropyl methacryliamide (INCI designation: polyquaternium-55) are also preferred and are available from the company ISP under brand names Styleze® W-10 or Styleze® W-20, for example.

Finally, the most especially preferred cationic polymers a3) are copolymers of N-vinylpyrrolidone, N-vinylcaprolactam, N-(3-dimethylaminopropyl)methacrylamide and 3-(methacryloylamino)propyllauryldimethyl ammonium chloride (INCI designation: polyquaternium-69), which are distributed by the company ISP, for example, under the brand name AquaStyle® 300 (28-32% by weight active substance in an ethanol-water mixture, molecular weight 350,000).

Because of their better-than-average styling properties, such cosmetic preparations a), which contain both nonionic polymers and cationic polymers a3) are preferred in particular.

The inventive cosmetic products also include a device for flash evaporation, in addition to cosmetic preparation a). Within the context of the present patent application, the term “flash evaporation” refers to the development of steam when the pressure is lowered in a closed space that is filled with fluid and is under an excess pressure (with respect to the environment). A corresponding excess pressure can be created, for example, by heating a quantity of the cosmetic preparation a) in a closed space to a temperature T₁. In the closed space, the fluid has a saturation pressure pi at a given temperature T₁. If the closed space is opened by employing a valve to a reaction space, which is not under an excess pressure, but has the pressure p₀<p₁, then the pressure in the previously closed space drops and the cosmetic preparation a) evaporates as part of the propagation of the new pressure level and/or the solvent or portions of this solvent contained in the cosmetic preparation evaporate(s). The resulting vapor or spray mist may be utilized for application of specific cosmetic preparations.

If the cosmetic preparation is thus heated in a closed space, starting from standard conditions (T₀=25° C., p₀=1000 bar), this results not only in an elevated temperature but also an elevated pressure of the cosmetic preparation a). This elevated pressure can be released in a relaxation space to a pressure po, for example, the ambient atmospheric pressure (p₀=1000 bar), so that at least partial evaporation of the cosmetic preparation a) is achieved.

The cosmetic preparation a) can be depressurized directly in the space in which it was previously heated. However, as an alternative, the heated cosmetic preparation a), which is under an excess pressure, may also be transported into a second space, where the pressure release then takes place subsequently.

In other words, flash evaporation is a method, in which the cosmetic preparation a) is heated in a closed container by employing a heating apparatus to temperatures above the ambient temperature, wherein a pressure above the ambient pressure develops in the container, and the heated cosmetic preparation a), which is then under an elevated pressure is released into the environment from the container.

A device for flash evaporation is thus a device comprising a container and a heating apparatus and is designed so that a cosmetic preparation a) can be heated in the closed container by employing the heating apparatus to temperatures above ambient temperature, such that a pressure above ambient pressure is created in the container, and the heated cosmetic preparation a), which is under pressure, can then be released from the container into the environment.

Simultaneously with or after the pressure release, the cosmetic preparation a) can be sent through a nozzle, such that properties of the vapor and/or spray mist created by the flash evaporation, in particular the droplet size or droplet density, but also the spray width and the shape of the spray cone can be influenced. The use of nozzles, preferably atomizer nozzles, is therefore preferred. The specific type of nozzle or the specific design of the nozzle is defined in a targeted manner as a function of the respective spray mist properties.

In summary, a preferred device for flash evaporation comprises
b1) a container b1), which is to be opened and closed by employing a valve, and which defines the closed interior space in which the cosmetic preparation can be accommodated,
b2) a heating apparatus b2), which makes it possible to heat a cosmetic preparation in the container b1).

Use of an additional nozzle b3), which permits atomization of the cosmetic preparation a) escaping from the container, is especially preferred. As an alternative to a valve, a closing element which has a comparable effect and can close or release a respective opening in the container through a corresponding change in position may also be used.

A preferred subject matter of the present disclosure is a cosmetic product, comprising:
a) a cosmetic preparation containing, based on its total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a), wherein the device for flash evaporation comprises a container b1) and a heating apparatus b2) and is designed, so that
the cosmetic preparation a) can be accommodated in the interior of the container b1),
the interior space of the container b1), which is filled at least proportionately with the cosmetic preparation a), can be closed,
the cosmetic preparation a) can be heated in the closed interior space of the container b1) by employing the heating apparatus b2) under an elevated pressure.

A particularly preferred subject matter of the present disclosure is therefore a cosmetic product, comprising
a) a cosmetic preparation containing, based on its total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve;
b2) a heating apparatus, which makes it possible to heat a cosmetic preparation in the container b1),
b3) a nozzle b3), which makes it possible to atomize the cosmetic preparation a).

Another preferred subject matter of the present disclosure is therefore a cosmetic product, comprising:
a) a cosmetic preparation containing, based on its total weight:
a1) about 45% to about 98% by weight of at least one polar solvent, wherein the amount by weight of water relative to the total weight of the polar solvent a1) preferably amounts to at least about 80% by weight, especially at least about 88% by weight, especially preferably at least about 92% by weight;
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve,
b2) a heating apparatus, which makes it possible to heat a cosmetic preparation in the container b1),
b3) a nozzle b3), which makes it possible to atomize the cosmetic preparation a).

In other words, an especially preferred subject matter of the present disclosure is a cosmetic product, comprising:
a) a cosmetic preparation containing, based on its total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a), wherein the device for flash evaporation comprises a container b1) and a heating apparatus b2) and is designed, so that
the cosmetic preparation a) can be accommodated in the interior of the container b1),
the interior space of the container b1), which is filled at least proportionately with the cosmetic preparation a), can be closed,
the cosmetic preparation a) can be heated in the closed interior space of the container b1) by employing the heating apparatus b2) under an increase in pressure,
the heated cosmetic preparation a) from the interior space of the container b1) can be released into the environment with a drop-in pressure.
The container b1), in which the cosmetic preparation is heated, is designed in such a way that makes it possible to completely seal this container with respect to the environment during the heating of the cosmetic preparation a), and then, after the heating, to open it in order to enable flash evaporation of the cosmetic preparation a). This can be assured by employing a component for regulating the flow, in particular a valve, for example.

The container b1), in which the cosmetic preparation is heated, is preferably in contact with another container, from which the amount of cosmetic preparation provided for flash evaporation is transferred into the container b1) before heating. The access between this storage container and container b1) is to be opened and closed by employing a suitable device, such as a valve. This additional container is preferably designed in the form of a storage container, i.e., it preferably comprises many times the amount of cosmetic preparation required for an evaporation process, for example, more than ten times, preferably more than fifty times that amount. In other words, the additional container/supply container preferably has a volume many times greater than the volume of container b1), for example, more than ten times greater, preferably more than twenty times greater and in particular more than fifty times greater than that of container b1).

Another especially preferred subject matter of the present disclosure is therefore a cosmetic product, comprising:
a) a cosmetic preparation, containing, based on its total weight:
a1) about 45% to about 98% by weight of at least one polar solvent,
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve,
b2) a heating apparatus, which makes it possible to heat a cosmetic preparation in the container b1),
b3) a nozzle b3), which makes it possible to atomize the cosmetic preparation a);
c) a storage container for the cosmetic preparation a), from which the cosmetic preparation a) can enter the container b1), wherein
the access between the storage container and container b1) has a component for regulating the flow such that the flow of the cosmetic preparation a) out of the storage container and into the container b1) can be interrupted;
the storage container has a volume at least ten times greater, preferably at least twenty times greater, and in particular at least about fifty times greater than the volume of container b1).

The storage container is not a pressurized container and the cosmetic preparation in the storage container is not under pressure. In other words, the pressure in the interior of the storage container corresponds to ambient pressure (also referred to as air pressure or atmospheric pressure). Thus, the corresponding cosmetic products do not include a blowing agent. The cosmetic product also does not have a pump device that is suitable for releasing or spraying the cosmetic preparation into the environment without the action of the device for flash evaporation.

A most especially preferred subject matter of the present disclosure is therefore a cosmetic product, comprising:
a) a cosmetic preparation containing, based on its total weight:
a1) about 45% to about 98% by weight of at least one polar solvent,
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve,
b2) a heating apparatus, which makes it possible to heat a cosmetic preparation in the container b1),
b3) a nozzle b3), which makes it possible to atomize the cosmetic preparation a).
c) a storage container for the cosmetic preparation a) from which the cosmetic preparation a) can enter the container b1), wherein
the access between the storage container and container b1) has a component for regulating the flow such that the flow of the cosmetic preparation a) out of the storage container and into the container b1) can be interrupted;
the storage container has a volume at least ten times greater, preferably at least twenty times greater, and in particular at least about fifty times greater than the volume of container b1);
the pressure in the interior of the storage container corresponds to the ambient pressure.

A most especially preferred subject matter of the present disclosure is therefore a cosmetic product, comprising:
a) a cosmetic preparation containing, based on its total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve,
b2) a heating apparatus, which makes it possible to heat a cosmetic preparation in the container b1),
b3) a nozzle b3), which makes it possible to atomize the cosmetic preparation a).
c) a storage container for the cosmetic preparation a) from which the cosmetic preparation a) can enter the container b1), wherein
the access between the storage container and container b1) has a component for regulating the flow such that the flow of the cosmetic preparation a) out of the storage container and into the container b1) can be interrupted;
the storage container has a volume at least ten times greater, and in particular at least fifty times greater than the volume of container b1);
the pressure in the interior of the storage container corresponds to the ambient pressure and the cosmetic product does not include a blowing agent.

In addition, cosmetic products comprising the following are preferred:
a) a cosmetic preparation containing, based on its total weight:
a1) about 45% to about 98% by weight of at least one polar solvent,
a2) about 0.1% to about 4.0% by weight chitosan;
a3) about 0.1% to about 15% by weight film-forming polymer;
b) a device for flash evaporation of the cosmetic preparation a), comprising:
b1) a container b1), which is to be opened and closed by employing a valve,
b2) a heating apparatus, which makes it possible to heat a cosmetic preparation in the container b1),
b3) a nozzle b3), which makes it possible to atomize the cosmetic preparation a).
c) a storage container for the cosmetic preparation a) from which the cosmetic preparation a) can enter the container b1), wherein
the access between the storage container and container b1) has a component for regulating the flow such that the flow of the cosmetic preparation a) out of the storage container and into the container b1) can be interrupted;
the storage container has a volume at least ten times greater, and in particular at least fifty times greater than the volume of container b1);
the pressure in the interior of the storage container corresponds to the ambient pressure, wherein the cosmetic product does not have a pump device suitable for releasing or spraying the cosmetic preparation a) without the action of the flash evaporation device.

In summary, an especially preferred subject matter of the present disclosure therefore is a cosmetic product, comprising
a) a cosmetic preparation containing, based on its total weight:
a1) about 45% to about 98% by weight of at least one polar solvent,
a2) about 0.1% to about 4.0% by weight chitosan;
a3) about 0.1% to about 15% by weight film-forming polymer;
b) a device for flash evaporation of the cosmetic preparation a), comprising
b1) a container b1), which is to be opened and closed by employing a valve, b2) a heating apparatus, which makes it possible to heat a cosmetic preparation in the container b1),
b3) a nozzle b3), which makes it possible to atomize the cosmetic preparation a);
c) a storage container for the cosmetic preparation a) from which the cosmetic preparation
a) can enter the container b1), wherein
the access between the storage container and container b1) has a component for regulating the flow such that the flow of the cosmetic preparation a) out of the storage container and into the container b1) can be interrupted;
the storage container has a volume at least ten times greater, and in particular at least fifty times greater than the volume of container b1);
the pressure in the interior of the storage container corresponds to the ambient pressure and the cosmetic product does not include a blowing agent,
wherein the cosmetic product does not have a pump device suitable for releasing or spraying the cosmetic preparation a) without the action of the flash evaporation device.

In addition to the two components a1), a2) and a3) described above, the cosmetic preparations a) as contemplated herein may also contain additional active ingredients or additives, wherein those active ingredients or additives, which improve the manufacturability, applicability and/or cosmetic effect of cosmetic preparations as contemplated herein are preferred in particular.

To improve the manufacturability, applicability and cosmetic effect, the cosmetic preparation a) preferably contains nonionic surfactant a4), wherein especially preferred cosmetic preparations a) are characterized in that they contain, based on their total weight about 0.05 to about 4.0% by weight, preferably about 0.1% to about 2.0% by weight and in particular about 0.2% to about 1.0% by weight nonionic surfactant a4).

Preferred nonionic surfactants or PEG derivatives of hydrogenated castor oil, which are obtainable, for example, under the designation PEG hydrogenated castor oil, for example, PEG-30 hydrogenated castor oil, PEG-33 hydrogenated castor oil, PEG-35 hydrogenated castor oil, PEG-36 hydrogenated castor oil, PEG-40 hydrogenated castor oil or PEG-60 hydrogenated castor oil. Especially preferred as contemplated herein are nonionic surfactants selected from the group of PEG derivatives of hydrogenated castor oil, especially preferably from the group comprised of PEG-40 hydrogenated castor oil and PEG-60 hydrogenated castor oil, in particular PEG-40 hydrogenated castor oil.

Another preferred component of the cosmetic preparations a) as contemplated herein is the cationic surfactants a5). Preferred cationic surfactants a5) are selected from quaternary ammonium compounds, ester quats and amidoamines. The cationic surfactants are present in the cosmetic preparation a) in amounts of about 0.01% to about 4.0% by weight, preferably about 0.05% to about 2.0% by weight, and in particular about 0.1% to about 1.0% by weight, based on their total weight. Cationic surfactants a5) from the group of quaternary ammonium compounds are especially preferred.

Preferred quaternary ammonium compounds include ammonium halides, in particular chlorides and bromides such as alkyltrimethylammonium chlorides, dialkyldimethylammonium chlorides and trialkylmethylammonium chlorides, for example, cetyltrimethylammonium chloride, stearyltrimethylammonium chloride, disteryldimethylammonium chloride, lauryldimethylammonium chloride, lauryldimethylbenzylammonium chloride and triacetylmethylammonium chloride, as well as the imidazolium compounds known under the INCI designations quaternium-27 and quaternium-83. The long alkyl chains of the aforementioned surfactants preferably have 10 to 18 carbon atoms. Most especially preferred cosmetic preparations a) as contemplated herein are characterized in that they contain, based on their total weight, about 0.01% to about 4.0% by weight, preferably about 0.05% to about 2.0% by weight and in particular about 0.1% to about 1.0% by weight (C₁₂ to C₁₈)-alkyltrimethylammonium salt(s).

Additional care substances can be mentioned in particular as further suitable active ingredients or auxiliary substances.

The agent may contain as the care substance, for example, at least one protein hydrolysate and/or one of its derivatives. Protein hydrolysates are product mixtures obtained by acidic, basic or enzymatically catalyzed degradation of proteins. The term “protein hydrolysates” as contemplated herein is also understood to refer to total hydrolysates as well as individual amino acids and their derivatives as well as mixtures of various amino acids. The molecular weight of the protein hydrolysates that can be used as contemplated herein is between about 75, the molecular weight for glycine, and about 200,000, preferably the molecular weight amounting to about 75 to about 50,000 and most especially preferably about 75 to about 20,000 Dalton.

As the care substance, the inventive agent may additionally contain at least one vitamin, one provitamin, one vitamin precursor and/or a derivative thereof. The vitamins, provitamins and vitamin precursors that are preferred as contemplated herein are usually assigned to the groups A, B, C, E, F and H.

Additional care substances include panthenol, caffeine, nicotinamide and sorbitol.

The inventive agents additionally contain as the care substance at least one plant extract but also mono- and/or oligosaccharides and/or lipids.

The composition of some especially preferred cosmetic preparations as contemplated herein may contain the following tables (amounts given in % by weight, based on the total weight of the cosmetic agent, unless otherwise indicated). With respect to additional preferred specific embodiments of these specially preferred compositions, what was said above regarding the cosmetic preparations a) as contemplated herein also applies here, mutatis mutandis.

| | Formula | Formula | Formula | Formula | Formula |
|---|---|---|---|---|---|
| | 1 | 2 | 3 | 4 | 5 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Film-forming | 0.1 to 15 | 1.0 to 12 | 2.0 to 10 | 3.9 | 8.9 |
| polymer a3) | | | | | |
| Optional | add 100 | add 100 | add 100 | add 100 | add 100 |
| additives | | | | | |

| | Formula | Formula | Formula | Formula | Formula |
|---|---|---|---|---|---|
| | 6 | 7 | 8 | 9 | 10 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyvinyl- | 0.1 to 15 | 1.0 to 12 | 2.0 to 10 | 3.9 | 8.9 |
| pyrrolidone | | | | | |
| Optional | add 100 | add 100 | add 100 | add 100 | add 100 |
| additives | | | | | |

| | Formula | Formula | Formula | Formula | Formula |
|---|---|---|---|---|---|
| | 11 | 12 | 13 | 14 | 15 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Vinylpyr- | 0.1 to 15 | 1.0 to 12 | 2.0 to 10 | 3.9 | 8.9 |
| rolidone/vinyl | | | | | |
| acetate | | | | | |
| copolymer | | | | | |
| Optional | add 100 | add 100 | add 100 | add 100 | add 100 |
| additives | | | | | |

| | Formula | Formula | Formula | Formula | Formula |
|---|---|---|---|---|---|
| | 16 | 17 | 18 | 19 | 20 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquater- | 0.1 to 15 | 1.0 to 12 | 2.0 to 10 | 3.9 | 8.9 |
| nium-4 | | | | | |
| Optional | add 100 | add 100 | add 100 | add 100 | add 100 |
| additives | | | | | |

| | Formula | Formula | Formula | Formula | Formula |
|---|---|---|---|---|---|
| | 21 | 22 | 23 | 24 | 25 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquater- | 0.1 to 15 | 1.0 to 12 | 2.0 to 10 | 3.9 | 8.9 |
| nium-11 | | | | | |
| Optional | add 100 | add 100 | add 100 | add 100 | add 100 |
| additives | | | | | |

| | Formula | Formula | Formula | Formula | Formula |
|---|---|---|---|---|---|
| | 26 | 27 | 28 | 29 | 30 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquater- | 0.1 to 15 | 1.0 to 12 | 2.0 to 10 | 3.9 | 8.9 |
| nium-16 | | | | | |
| Optional | add 100 | add 100 | add 100 | add 100 | add 100 |
| additives | | | | | |

| | Formula | Formula | Formula | Formula | Formula |
|---|---|---|---|---|---|
| | 31 | 32 | 33 | 34 | 35 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 55 | | | | | |
| Optional additives | add 100 | add 100 | add 100 | add 100 | add 100 |

| | Formula | Formula | Formula | Formula | Formula |
|---|---|---|---|---|---|
| | 36 | 37 | 38 | 39 | 40 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 69 | | | | | |
| Optional additives | add 100 | add 100 | add 100 | add 100 | add 100 |

| | Formula | Formula | Formula | Formula | Formula |
|---|---|---|---|---|---|
| | 41 | 42 | 43 | 44 | 45 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Combination of | 0.1 to 15 | 1.0 to 12 | 2.0 to 10 | 3.9 | 8.9 |
| nonionic and | | | | | |
| cationic film- | | | | | |
| forming polymers | | | | | |
| a3) | | | | | |
| Optional additives | add 100 | add 100 | add 100 | add 100 | add 100 |

| | Formula | Formula | Formula | Formula | Formula |
|---|---|---|---|---|---|
| | 46 | 47 | 48 | 49 | 50 |
| Polar solvent | 45 to 98 | 55 to 95 | 70 to 92 | 94 | 90 |
| a1) | | | | | |
| Chitosan a2) | 0.1 to 4.0 | 0.1 to 2.0 | 0.2 to 1.0 | 0.4 | 0.6 |
| Film-forming | 0.1 to 15 | 1.0 to 12 | 2.0 to 10 | 3.9 | 8.9 |
| polymer a3) | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | | Formula | |
|---|---|---|---|---|---|
| | 51 | 52 | Formula 53 | 54 | Formula 55 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyvinylpyrrolidone | 0.1 to 15 | 1.0 to 12 | 2.0 to 10 | 3.9 | 8.9 |
| Nonionic surfactant a4) | 0.05 to 4.0 | 0.1 to 2.0 | 0.2 to 1.0 | 0.2 | 0.2 |
| Optional additives | add 100 | add 100 | add 100 | add 100 | add 100 |

| | Formula | Formula | Formula | Formula | Formula |
|---|---|---|---|---|---|
| | 56 | 57 | 58 | 59 | 60 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Vinylpyrrolidone/ | 0.1 to 15 | 1.0 to 12 | 2.0 to 10 | 3.9 | 8.9 |
| vinyl acetate | | | | | |
| copolymer | | | | | |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 61 | 62 | 63 | 64 | 65 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium-4 | 0.1 to 15 | 1.0 to 12 | 2.0 to 10 | 3.9 | 8.9 |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 66 | 67 | 68 | 69 | 70 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 11 | | | | | |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 71 | 72 | 73 | 74 | 75 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 16 | | | | | |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 76 | 77 | 78 | 79 | 80 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 55 | | | | | |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 81 | 82 | 83 | 84 | 85 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyquaternium- | 0.1 to 15 | 1.0 to 12 | 2.0 to 10 | 3.9 | 8.9 |
| 69 | | | | | |
| Nonionic | 0.05 to 4.0 | 0.1 to 2.0 | 0.2 to 1.0 | 0.2 | 0.2 |
| surfactant a4) | | | | | |
| Optional additives | add 100 | add 100 | add 100 | add 100 | add 100 |

| | Formula | Formula | Formula | Formula | Formula |
|---|---|---|---|---|---|
| | 86 | 87 | 88 | 89 | 90 |
| Polar solvent a1) | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Combination of | 0.1 to 15 | 1.0 to 12 | 2.0 to 10 | 3.9 | 8.9 |
| nonionic and | | | | | |
| cationic | | | | | |
| film-forming | | | | | |
| polymers a3) | | | | | |
| Nonionic | 0.05 to 4.0 | 0.1 to 2.0 | 0.2 to 1.0 | 0.2 | 0.2 |
| surfactant | | | | | |
| a4) | | | | | |
| Optional additives | add 100 | add 100 | add 100 | add 100 | add 100 |

| | Formula | Formula | Formula | Formula | Formula |
|---|---|---|---|---|---|
| | 91 | 92 | 93 | 94 | 95 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Film-forming | 0.1 to 15 | 1.0 to 12 | 2.0 to 10 | 3.9 | 8.9 |
| polymer a3) | | | | | |
| Optional additives | add 100 | add 100 | add 100 | add 100 | add 100 |

| | | Formula | | Formula | |
|---|---|---|---|---|---|
| | Formula 96 | 97 | Formula 98 | 99 | Formula 100 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyvinylpyrrolidone | 0.1 to 15 | 1.0 to 12 | 2.0 to 10 | 3.9 | 8.9 |
| Optional additives | add 100 | add 100 | add 100 | add 100 | add 100 |

| | Formula | Formula | Formula | Formula | Formula |
|---|---|---|---|---|---|
| | 101 | 102 | 103 | 104 | 105 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Vinylpyrrolidone/ | 0.1 to 15 | 1.0 to 12 | 2.0 to 10 | 3.9 | 8.9 |
| vinyl acetate | | | | | |
| copolymer | | | | | |
| Optional additives | add 100 | add 100 | add 100 | add 100 | add 100 |

| | Formula | Formula | Formula | Formula | Formula |
|---|---|---|---|---|---|
| | 106 | 107 | 108 | 109 | 110 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-4 | | | | | |
| Optional | add 100 | add 100 | add 100 | add 100 | add 100 |
| additives | | | | | |

| | Formula | Formula | Formula | Formula | Formula |
|---|---|---|---|---|---|
| | 111 | 112 | 113 | 114 | 115 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-11 | | | | | |
| Optional | add 100 | add 100 | add 100 | add 100 | add 100 |
| additives | | | | | |

| | Formula | Formula | Formula | Formula | Formula |
|---|---|---|---|---|---|
| | 116 | 117 | 118 | 119 | 120 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-16 | | | | | |
| Optional | add 100 | add 100 | add 100 | add 100 | add 100 |
| additives | | | | | |

| | Formula | Formula | Formula | Formula | Formula |
|---|---|---|---|---|---|
| | 121 | 122 | 123 | 124 | 125 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-55 | | | | | |
| Optional | add 100 | add 100 | add 100 | add 100 | add 100 |
| additives | | | | | |

| | Formula | Formula | Formula | Formula | Formula |
|---|---|---|---|---|---|
| | 126 | 127 | 128 | 129 | 130 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-69 | | | | | |
| Optional | add 100 | add 100 | add 100 | add 100 | add 100 |
| additives | | | | | |

| | Formula | Formula | Formula | Formula | Formula |
|---|---|---|---|---|---|
| | 131 | 132 | 133 | 134 | 135 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Combination of | 0.1 to 15 | 1.0 to 12 | 2.0 to 10 | 3.9 | 8.9 |
| nonionic and | | | | | |
| cationic film- | | | | | |
| forming | | | | | |
| polymers a3) | | | | | |
| Optional | add 100 | add 100 | add 100 | add 100 | add 100 |
| additives | | | | | |

| | Formula | Formula | Formula | Formula | Formula |
|---|---|---|---|---|---|
| | 136 | 137 | 138 | 139 | 140 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Film-forming | 0.1 to 15 | 1.0 to 12 | 2.0 to 10 | 3.9 | 8.9 |
| polymer a3) | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 141 | 142 | 143 | 144 | 145 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Polyvinyl- | 0.1 to 15 | 1.0 to 12 | 2.0 to 10 | 3.9 | 8.9 |
| pyrrolidone | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 146 | 147 | 148 | 149 | 150 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Vinylpyr- | 0.1 to 15 | 1.0 to 12 | 2.0 to 10 | 3.9 | 8.9 |
| rolidone/vinyl | | | | | |
| acetate | | | | | |
| copolymer | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 151 | 152 | 153 | 154 | 155 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-4 | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 156 | 157 | 158 | 159 | 160 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-11 | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 161 | 162 | 163 | 164 | 165 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-16 | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 166 | 167 | 168 | 169 | 170 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-55 | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 171 | 172 | 173 | 174 | 175 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Poly- | 0.1 to 15 | 1.0 to 12 | 2.0 to 10 | 3.9 | 8.9 |
| quaternium-69 | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

| | Formula | Formula | Formula | Formula | Formula |
|---|---|---|---|---|---|
| | 176 | 177 | 178 | 179 | 180 |
| Water | 45 to 98 | 55 to 95 | 70 to 92 | 94 | 90 |
| Chitosan a2) | 0.1 to 4.0 | 0.1 to 2.0 | 0.2 to 1.0 | 0.4 | 0.6 |
| Combination of | 0.1 to 15 | 1.0 to 12 | 2.0 to 10 | 3.9 | 8.9 |
| nonionic and | | | | | |
| cationic film- | | | | | |
| forming | | | | | |
| polymers a3) | | | | | |
| Nonionic | 0.05 to 4.0 | 0.1 to 2.0 | 0.2 to 1.0 | 0.2 | 0.2 |
| surfactant a4) | | | | | |
| Optional | add 100 | add 100 | add 100 | add 100 | add 100 |
| additives | | | | | |

Most especially preferred cosmetic preparations contain, in addition to the ingredients a1) through a5) described above, only small amounts of other active ingredients and additives. Cosmetic preparations that are characterized in that the amount by weight of ingredients a1), a2) and, if present, a3) and/or a4) and/or a5) in the total weight of the cosmetic preparation add up to at least about 80% by weight, preferably at least about 90% by weight, especially preferably at least about 93% by weight and in particular at least about 97% by weight are especially preferred because they are simple to produce and have a good cosmetic effect. The most especially preferred cosmetic preparations consist of at least about 80% by weight, preferably at least about 87% by weight, and in particular at least about 95% by weight of ingredients a1), a2) and a3), based on their total weight.

As explained in the introduction, the cosmetic preparations a) as contemplated herein are suitable in particular for application by employing a device for flash evaporation. Another subject matter of the present disclosure is therefore the use of a cosmetic preparation a) containing, based on its total weight:
a1) about 45% to about 98% by weight of at least one polar solvent;
a2) about 0.1% to about 4.0% by weight chitosan;
a3) about 0.1% to about 15% by weight film-forming polymer, as the process material in a device for flash evaporation.

The subject matter of the present patent application is also the use of a product as contemplated herein for application to keratin-containing fibers, in particular human hair, for applying a cosmetic preparation a) and/or for temporary shaping of keratin-containing fibers, in particular human hair.

A method for temporary shaping of keratin-containing fibers, in particular human hair, in which the keratin-containing fibers are treated by employing a device for flash evaporation with a cosmetic preparation a) containing, based on their total weight:
a1) about 45% to about 98% by weight of at least one polar solvent;
a2) about 0.1% to about 4.0% by weight chitosan;
a3) about 0.1% to about 15% by weight film-forming polymer, this method is another subject matter of the present patent application. The cosmetic preparation a) is preferably converted to a spray mist by the device for flash evaporation and then this spray mist is then used to treat the keratin-containing fibers.

To achieve an adequate spray effect, the cosmetic preparation a) is preferably heated to temperatures above the boiling point of the polar solvent or solvent mixture contained in the cosmetic preparation a).

If the polar solvent here is water or a solvent mixture with a water content of more than about 50% by weight (based on the total weight of the solvent mixture), then the cosmetic preparation is preferably heated to temperatures above about 100° C., preferably to temperatures of about 100° C. to about 240° C., especially preferably to temperatures of about 140° C. to about 160° C.

The excess pressure achieved due to the heating of the cosmetic preparation a) preferably amounts to between about 1.1 and about 8 bar, preferably between about 1.2 and about 4 bar in those cases in which the polar solvent is water or a solvent mixture with a water content of more than about 50% by weight (based on the total weight of the solvent mixture).

A preferred subject matter of the patent application is a method for changing the color of keratin-containing fibers, in particular human hair, in which the keratin-containing fibers are treated, by using a device for flash evaporation, with a cosmetic preparation a) containing, based on the total weight of each:
a1) about 45% to about 98% by weight of at least one polar solvent;
a2) about 0.1% to about 4.0% by weight chitosan;
a3) about 0.1% to about 15% by weight film-forming polymer;
wherein
a partial amount of the cosmetic preparation a), which is in a storage container, in the interior of which a pressure corresponding to the ambient pressure prevails, is transferred from that storage container into a container b1);
next, the access between the storage container and container b1) is interrupted by a component for flow control, such that the flow of the cosmetic preparation a) out of the storage container and into the container b1) can be interrupted;
next, the cosmetic preparation a), which is in the container b1) that is sealed off with respect to the environment, is heated by employing a heating apparatus, so that the pressure inside the container b1) rises to levels above ambient pressure, preferably to levels between about 1.1 and about 8 bar, in particular to levels between about 1.2 and about 4 bar;
next the container b1), which is under a higher pressure than the ambient pressure, is opened in such a way that it depressurizes the outlet of at least a partial amount, preferably at least 50% by weight, preferably at least 80% by weight and in particular at least 90% by weight of the cosmetic preparation in the container b1) out of the container b1) into the environment with a reduction in the pressure prevailing at the point in time of the container opening into the container b1).

The depressurization of the cosmetic preparation a) into the environment preferably takes place with the formation of a spray mist of the cosmetic preparation a).

The cosmetic preparation a), which is flash-evaporated out of the container b1), is preferably applied to keratinic fibers, in particular to human hair.

Methods in the course of which the cosmetic preparation depressurized out of the container b1) is sent through a nozzle before treating the keratinic fibers, are especially preferred.

With respect to other preferred specific embodiments of the applications as contemplated herein uses and the method as contemplated herein, what was said above regarding the cosmetic preparations a) as contemplated herein and the device for flash evaporation is also true here mutatis mutandis.

In summary, the agents, applications and methods as contemplated herein and some of their preferred specific embodiments are characterized by the following points:
1. A cosmetic product, comprising:
a) cosmetic preparation containing, based on their total weight:
a1) about 45% to about 98% by weight of at least one polar solvent;
a2) about 0.1% to about 4.0% by weight chitosan;
a3) about 0.1% to about 15% by weight film-forming polymer;
b) a device for flash evaporation of the cosmetic preparation a).
2. The cosmetic product according to point 1, characterized in that the device for flash evaporation comprises a container b1) and a heating apparatus b2) and is designed so that
the cosmetic preparation a) can be accommodated in the interior space of the container b1),
the interior space of the container b1), which is filled at least proportionately with the cosmetic preparation a) can be sealed,
the cosmetic preparation a) can be heated in the closed interior space of the container b1) by employing the heating apparatus b2) with an increase in pressure,
the heated cosmetic preparation a) can be flash-evaporated out of the interior space of the container b1) and into the environment with a drop-in pressure.
3. The cosmetic product according to any one of the preceding points, characterized in that the amount by weight of the polar solvent a1) relative to the total weight of the cosmetic preparation a) amounts to about 55% to about 95% by weight, preferably about 70% to about 92% by weight.
4. The cosmetic product according to any one of the preceding points, characterized in that the polar solvent a1) is at a boiling point (20° C., 1013 mbar) between about 50° C. and about 110° C., preferably between about 70° C. and about 105° C.
5. The cosmetic products according to any one of the preceding points, characterized in that the polar solvent a1) is selected from the group comprising ethanol, isopropanol and water.
6. The cosmetic product according to any one of the preceding points, characterized in that the amount by weight of water and ethanol relative to the total weight of the polar solvent a1) amounts to at least about 60% by weight, preferably at least about 80% by weight, especially preferably at least about 90% by weight and in particular at least about 95% by weight.
7. The cosmetic product according to any one of the preceding points, characterized in that the amount by weight of water relative to the total weight of the polar solvent a1) amounts to more than about 80% by weight, preferably more than about 88% by weight, and in particular more than about 92% by weight.
8. The cosmetic product according to any one of the preceding points, characterized in that the amount by weight of the ethanol relative to the total weight of the cosmetic preparation amounts to no greater than 55% by weight, preferably about 10% to about 55% by weight, especially preferably about 25% to about 55% by weight and in particular about 40% to about 55% by weight.
9. The cosmetic product according to any one of the preceding points, characterized in that the amount by weight of chitosan a2) relative to the total weight of the cosmetic preparation a) amounts to about 0.1% to about 2.0% by weight, preferably about 0.2% to about 1.0% by weight.
10. The cosmetic product according to any one of the preceding points, characterized in that the preparation a) contains, based on its total weight, about 1.0% to about 12% by weight, preferably about 2.0% to about 10% by weight film-forming polymer a3).
11. The cosmetic product according to point 10, characterized in that the film-forming polymer a3) is selected from the group of nonionic polymers, preferably from the group of polyvinylpyrrolidones and polyvinylpyrrolidone/vinyl acetate copolymers, preferably the polyvinylpyrrolidone/vinyl acetate copolymers.
12. The cosmetic product according to point 10, characterized in that the film-forming polymer a3) is selected from the group of cationic polymers, preferably from the group of cationic polymers with the INCI designations polyquaternium-4, polyquaternium-11, polyquaternium-16, polyquaternium-55, polyquaternium-69.
13. The cosmetic product according to any one of the preceding points, characterized in that the cosmetic preparation a) contains, based on its total weight, about 0.05% to about 4.0% by weight, preferably about 0.1% to about 2.0% by weight and in particular about 0.2% to about 1.0% by weight nonionic surfactant a4).
14. The cosmetic product according to point 13, characterized in that the nonionic surfactant is selected from the group of PEG derivatives of hydrogenated castor oil, especially preferably from the group of PEG-40 hydrogenated castor oil and PEG-60 hydrogenated castor oil.
15. The cosmetic product according to any one of the preceding points, characterized in that the cosmetic preparation a) contains, based on its total weight, about 0.05 to about 3.0% by weight, preferably about 0.1% to about 2.0% by weight and in particular about 0.2% to about 1.0% by weight cationic surfactant a5).
16. The cosmetic product according to point 15, characterized in that the cationic surfactant is selected from the groups of quaternary ammonium compounds, ester quats and amidoamines, preferably from the group of quaternary ammonium compounds, in particular from the group of (C₁₂ to C₁₈)-alkyltrimethyl ammonium salts.
17. The cosmetic product according to any one of the preceding points, characterized in that the amount by weight of ingredients a1), a2) and a3) relative to the total weight of the cosmetic preparation amounts to at least about 80% by weight, preferably at least about 87% by weight and in particular at least about 95% by weight.
18. A use of a cosmetic preparation a) containing, based on its total weight,
a1) about 45% to about 98% by weight of at least one polar solvent;
a2) about 0.1% to about 4.0% by weight chitosan;
a3) about 0.1% to about 15% by weight film-forming polymer, as the process material in a device for flash evaporation.
19. The use of a product according to any one of points 1 to 17 for treating keratin-containing fibers, in particular human hair, with a cosmetic preparation a).
20. The use of a product according to points 1 to 17 for temporary shaping of keratin-containing fibers, in particular human hair.
21. A method for temporary shaping of keratin-containing fibers, in particular human hair, in which the keratin-containing fibers are treated by employing a device for flash evaporation, with a cosmetic preparation a) containing, based on its total weight
a1) about 45% to about 98% by weight of at least one polar solvent;
a2) about 0.1% to about 4.0% by weight chitosan;
a3) about 0.1% to about 15% by weight film-forming polymer.
22. The method according to point 21, characterized in that
from a storage container in whose interior a pressure corresponding to ambient pressure prevails, a partial amount of the cosmetic preparation a) contained in this storage container is transferred to a container b1);
then the access between the storage container and container b1) is interrupted by a component for flow control such that the flow of cosmetic preparation a) out of the storage container and into container b1) can be interrupted;
then the cosmetic preparation a) present in the container b1) that has been sealed with respect to the environment is heated by employing a heating apparatus, so that the pressure in the interior of the container b1) rises to levels above ambient pressure, preferably to levels between about 1.1 and about 8 bar, in particular to levels between about 1.2 and about 4 bar;
then the container b1), which is under a pressure greater than ambient pressure, is opened in such a way that it depressurizes the outlet of at least a partial amount, preferably at least about 50% by weight, especially at least about 80% by weight and in particular at least about 90% by weight of the cosmetic preparation contained in the container b1) out of the container b1) and into the environment, with a drop in the pressure prevailing in the container b1) at the point in time when the container is opened.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product, comprising:
a) a cosmetic preparation, comprising, based on its total weight;
a1) about 45% to about 98% by weight of at least one polar solvent;
a2) about 0.1% to about 4.0% by weight chitosan;
a3) about 0.1% to about 15% by weight film-forming polymer; and
b) a device for flash evaporation of the cosmetic preparation a).

2. The cosmetic product according to claim 1, wherein the device for flash evaporation comprises a container b1) and a heating apparatus b2) and is designed so that
the cosmetic preparation a) can be accommodated in the interior space of the container b 1),
the interior space of the container b1), which is filled at least proportionately with the cosmetic preparation a) can be sealed,
the cosmetic preparation a) can be heated in the closed interior space of the container b1) by employing the heating apparatus b2) with an increase in pressure, and
the heated cosmetic preparation a) can be flash-evaporated into the environment and out of the interior space of the container b1) with a drop in pressure.

3. The cosmetic product according to claim 1, wherein the amount by weight of the polar solvent a1) relative to the total weight of the cosmetic preparation a) amounts to about 55% to about 95% by weight.

4. The cosmetic product according to claim 1, wherein the amount by weight of chitosan a2) relative to the total weight of the cosmetic preparation a) amounts to about 0.1% to about 2.0% by weight.

5. The cosmetic products according to claim 1, wherein the preparation a) contains, based on its total weight, about 1.0% to about 12% by weight of the film-forming polymer a3).

6. The cosmetic product according to claim 4, wherein the film-forming polymer a3) is selected from the group of nonionic polymers.

7. The cosmetic product according to claim 4, wherein the film-forming polymer a3) is selected from the group of cationic polymers.

8. A cosmetic preparation a) comprising, based on its total weight,
a1) about 45% to about 98% by weight of at least one polar solvent;
a2) about 0.1% to about 4.0% by weight chitosan; and
a3) about 0.1% to about 15% by weight film-forming polymer;
wherein the cosmetic preparation a) is utilized as a material in a device for flash evaporation.

9. The cosmetic product according to claim 1, wherein the cosmetic product is utilized for treating keratin-containing fibers with a cosmetic preparation a).

10. The cosmetic product according to claim 1, wherein the cosmetic product is utilized for temporary shaping of keratin-containing fibers.

11. A method for temporary shaping of keratin-containing fibers, the method comprising: treating the keratin-containing fibers with a cosmetic preparation a) by employing a device for flash evaporation, the cosmetic preparation a) comprising, based on its total weight:
a1) about 45% to about 98% by weight of at least one polar solvent;
a2) about 0.1% to about 4.0% by weight chitosan; and
a3) about 0.1% to about 15% by weight film-forming polymer.

12. The method according to claim 11, wherein
from a storage container, in the interior of which a pressure corresponding to ambient pressure prevails, a partial amount of the cosmetic preparation a) contained in this storage container is transferred to a container b1);
then the access between the storage container and container b1) is interrupted by a component for flow control such that the flow of cosmetic preparation a) out of the storage container and into container b1) can be interrupted;
then the cosmetic preparation a) present in the container b1) that has been sealed with respect to the environment is heated by employing a heating apparatus, so that the pressure in the interior of the container b1) rises to levels above ambient pressure.
then the container b1), which is under a pressure greater than ambient pressure is opened such that it depressurizes the outlet of at least a partial amount of the cosmetic preparation contained in the container b1) out of the container b1) and into the environment with a drop in the pressure prevailing in the container b1) at the point in time when the container is opened.

13. The cosmetic product according to claim 6, wherein the film-forming polymer a3) is selected from the group of polyvinylpyrrolidones, polyvinylpyrrolidone/vinyl acetate copolymers, and combinations thereof.

14. The cosmetic product according to claim 13, wherein the film-forming polymer a3) is the polyvinylpyrrolidone/vinyl acetate copolymers.

15. The cosmetic product according to claim 7, wherein the film-forming polymer a3) is selected from the group of cationic polymers with the INCI designations polyquaternium-4, polyquaternium-11, polyquaternium-16, polyquaternium-55, polyquaternium-69, and combinations thereof.

16. The cosmetic product according to claim 1, wherein the cosmetic preparation a) comprises, based on its total weight, about 0.05% to about 4.0% by weight of a nonionic surfactant a4).

17. The cosmetic product according to claim 16, wherein the nonionic surfactant is selected from the group of PEG derivatives of hydrogenated castor oil.

18. The cosmetic product according to claim 1, wherein the cosmetic preparation a) comprises, based on its total weight, about 0.05 to about 3.0% by weight of a cationic surfactant a5).

19. The cosmetic product according to claim 18, wherein the cationic surfactant is selected from the groups of quaternary ammonium compounds, ester quats, amidoamines, and combinations thereof.

20. The cosmetic product according to claim 1, wherein the polar solvent a1) is selected from the group of ethanol, isopropanol, water, and combinations thereof.
